(19) United States
(12) Patent Application Publication
(10) Pub. No.: US 2019/0355461 A1
(43) Pub. Date: Nov. 21, 2019

(54) PRESCRIPTION MEDICATION DIVERSION ANALYSIS AND REPORTING

(71) Applicant: HENRY FORD HEALTH SYSTEM, Detroit, MI (US)

(72) Inventors: Sanjeev KUMAR, Ann Arbor, MI (US); James S. KALUS, Grosse Pointe Woods, MI (US); Edward G. SZANDZIK, Grosse Pointe Woods, MI (US)

(21) Appl. No.: 16/418,119

(22) Filed: May 21, 2019

Related U.S. Application Data
(60) Provisional application No. 62/674,059, filed on May 21, 2018.

(51) Int. Cl. G16H 20/10; G16H 10/60; G16H 50/50

(52) U.S. Cl. CPC G16H 20/10; G16H 50/50; G16H 10/60

(57) ABSTRACT

Medication administration records (MAR) risk data, entropy risk data, and automated dispensing risk data is generated from MAR records. A hybrid diversion risk is determined according to a weighing of the MAR risk data, the entropy risk data, and the automated dispensing risk data. Summaries of the hybrid diversion risk are precomputed. A visualization of the risk data is provided according to the precomputed summaries.

### CROSS-REFERENCE TO RELATED APPLICATIONS

This application claims the benefit of U.S. provisional application Ser. No. 62/674,059 filed May 21, 2018, the disclosure of which is hereby incorporated in its entirety by reference herein.

### TECHNICAL FIELD

Aspects of the disclosure generally relate to a software tool for analysis of reporting of prescription medication diversion.

### BACKGROUND

Healthcare providers are at a higher risk of drug abuse because of ease of access and high availability. The American Nurses Association estimates that six to eight percent of nurses use alcohol or drugs to an extent that is sufficient to impair professional performance. Other estimates suggest the misuse of drugs and alcohol at ten to fifteen percent. Although the rate is one out of ten in healthcare, the number of nurse providers that are disciplined is a minor fraction. Lack of adequate diversion monitoring solutions, evidence of diversion activities, and absence of proactive monitoring leads to identification and detection of only a handful of diversion activities.

### SUMMARY

In one or more illustrative embodiments, medication administration records (MAR) risk data, entropy risk data, and automated dispensing risk data is generated from MAR records. A hybrid diversion risk is determined according to a weighing of the MAR risk data, the entropy risk data, and the automated dispensing risk data. Summaries of the hybrid diversion risk are precomputed. A visualization of the risk data is provided according to the precomputed summaries.

### BRIEF DESCRIPTION OF THE DRAWINGS

FIG. 1 illustrates an example system including an artificially-intelligent (AI) digital platform to monitor and provide actionable intelligence around drug diversion activities in healthcare systems;

FIG. 2 illustrates an example structure of the AI platform;

FIG. 3 illustrates an example set of application components that implement various functionality of the AI platform;

FIG. 4 illustrates an example user interface of the AI platform providing a menu for actionable intelligence;

FIG. 5 illustrates an example user interface of the AI platform providing a graph of a system summary;

FIG. 6 illustrates an example user interface of the AI platform providing hybrid diversion information for a provider;

FIG. 7 illustrates an example user interface of the AI platform providing automated dispensing system risk information for a provider; and

FIG. 8 illustrates an example process for monitoring and providing actionable intelligence around drug diversion activities in healthcare systems.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Most current solutions for tracking diversion of medication target specific parts of an entire chain of events that occur when a drug is ordered for patient care. The standard norm for monitoring diversions is utilizing software at the point of dispensing controlled substances from automated dispensing cabinets. While this may help uncover instances of diversions, it is by no means a solution that covers the entire spectrum of possible avenues a diverter has at their behest.

Possible avenues for diversion of drugs include withdrawing drugs out of automated dispensing cabinets without any documented administration to the patient; withdrawing a larger than necessary dose and subsequent failure to adequately dispose of the drug as waste; accessing controlled substances as often as allowed by order, even when a patient is not in need of it; delays in administration of medications; or failure to administer medications.

A source of potential identification of possible diversion is medication administration records (MAR). Every clinical unit/floor in the hospital has highly variant patient acuity and thus highly-varying MAR. Thus, this varying acuity needs to be accounted for in the solution to avoid flagging false positives. It also means an application with a threshold-based system for identifying diversion instances will not succeed. Therefore, in order to analyze MAR, solutions needs to be cognitive of standard floor practice and intuitively adjust threshold as needed.

In addition to identifying potential diverters, a system needs to furbish evidence in the form of dispense with missing documentation on administration records. Therefore, a solution that aims to cover all of these varying aspects needs to be comprehensive and cohesive along the full spectrum of dispense to administration/diversion.

An artificially intelligent digital platform may be used to monitor and provide actionable intelligence around drug diversion activities in healthcare systems. With minimal intervention, the platform may be able to investigate diversion by studying dispense and medication administration records to find anomalous patterns.

The platform integrates a select array of technology pieces that are carefully knit together from years of experience working in a large healthcare system pharmacy. Designed from the ground up with multiple iterations and continuous testing and optimization, it pieces together the full picture view necessary to identify drug diversion.

The core of the platform utilizes deep learning technology. Specifically, using a family of self-supervised learning methods (sometime also referred to as unsupervised machine learning) known as autoencoders. At a high level, autoencoders are capable of learning the hidden patterns from the data with a relatively high precision. Autoencoders fall under deep learning since they utilize one or more hidden layers to either project the data in higher or lower than the input dimensions/features. Primarily, autoencoders have been utilized as precursors to larger neural networks to denoise the data or auto label the samples.

The platform uses autoencoders to learn patterns in high dimensional datasets. For example, all controlled substances administered by a nurse provider on a clinical floor are transposed as features which are fed into the pipeline to specifically identify the drugs that are more likely to be diverted by the diverting nurse.

The platform does so by projecting the input data into a lower dimensional subspace. The neural network is forced to squeeze the dimensions and learn only the most important features in the input data. This network model later reconstructs the data it identified in the first place. Since the platform forces the network to only learn the most important information about the data, the platform avoids learning the location of outliers with the same extent of accuracy. The platform can then measure this reconstruction error and label the data points that were most likely to be outliers.

Not only is the network, at this point, only able to answer who is the outlier in the high-dimensional dataset, it can also accurately identify the subspaces where the outlier is located. The platform may use this information to identify the dimensions in this subspace. If applied in the context of the earlier mentioned example, this would indicate, via the platform, the specific medications that a nurse is farther away from normal as compared peers that work similar shifts on the same floor.

Utilizing the strength of deep neural networks, the platform creates risk profiles of nurse providers at every point in the medication order. These risk scores are aggregated to identify a potential diverter along with the risk profiles where they scored highest. The platform models the data along a timeline to adjust for the temporal variation in diversion. The platform also looks into individual nurse provider's personal medication administration records (MAR) history to identify discrepancies related to relative affinity for specific medications. These individualized profiles are further matched across the clinical floor. Using natural language processing, the platform matches the free text comments on dispense overrides to nurse employee names and creates discrete fields. This data is then made available as evidence for further investigation along with all the risk profiles on a timeline. Thus, the platform is able to investigate diversion activity at scale for the entire health system or at an individual nurse-provider level.

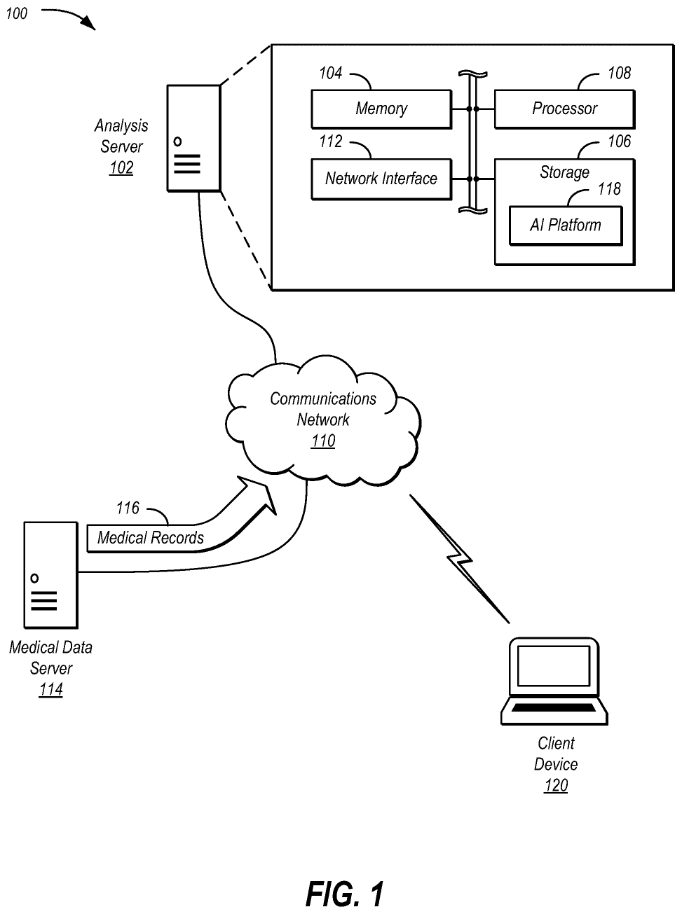

FIG. 1 illustrates an example system 100 including an artificially-intelligent digital platform 118 to monitor and provide actionable intelligence around drug diversion activities in healthcare systems. As illustrated, the system 100 includes an analysis server 102 hosting an artificial intelligence (AI) platform 118. The system 100 further includes a medical data server 114 hosting a secure data store maintaining MARs 116. The analysis server 102 may be configured to access the data server 114 over a communications network 110. The analysis server 102 may be further configured to maintain a portal accessible to client devices 120 accessible over the communication network 110. As explained in detail below, the system 100 may be configured to facilitate the analysis of the MARs 116 to identify drug diversion activities, and provide reporting of those activities to the client devices 120.

The analysis server 102 may include various types of computing apparatus, such as a computer workstation, a server, a desktop computer, a virtual server instance executed by a mainframe server, or some other computing system and/or device. Computing devices, such as the analysis server 102, generally include a memory 104 to which computer-executable instructions and data may be loaded from a storage 106, where the instructions may be executable by one or more processors 108 of the computing device. Such instructions and other data may be stored using a variety of computer-readable media. The computer-readable storage 106 (also referred to as a processor-readable medium or storage) includes any non-transitory (e.g., tangible) medium that participates in providing data (e.g., instructions) that may be read by a computer (e.g., by the processor 108 of the analysis server 102). In general, processors 108 receive instructions, e.g., from the memory 104 via the computer-readable storage medium 106 and execute these instructions, thereby performing one or more processes, including one or more of the processes described herein. Computer-executable instructions may be compiled or interpreted from computer programs created using a variety of programming languages and/or technologies, including, without limitation, and either alone or in combination, JAVA, C, C++, C#, FORTRAN, PASCAL, VISUAL BASIC, JAVA SCRIPT, PERL, PL/SQL, etc.

The communications network 110 may include one or more interconnected communication networks such as the Internet, a cable television distribution network, a satellite link network, a local area network, a wide area network, and a telephone network, as some non-limiting examples. The analysis server 102 may include a network interface 112 facilitating access by the analysis server 102 to the communications network 110.

Similar to as discussed above with respect to the analysis server 102, the medical data server 114 may include various types of computing apparatus (not shown) including a memory on which computer-executable instructions may be maintained, where the instructions may be executable by one or more processors of the computing device. The medical data server 114 may be configured to maintain MARs 116 to be analyzed by the analysis server 102. The MARs 116 may include information related to medications prescribed to patients. For example, the MARs 116 may indicate counts of individual medications prescribed, the identities of the medications that were prescribed, and the identity of the nurse or other medical professional who administered the medication.

The AI platform 118 may be an application included on the storage 106 of the analysis server 102. The AI platform 118 may include instructions that, when loaded into the memory 104 and executed by the processor 108 of the analysis server 102, cause the analysis server 102 to perform operations discussed in detail herein.

The client devices 120 may include various devices usable to access the AI platform 118 provided by the analysis server 102 over the communications network 110. The client devices 120 may include laptop computers, tablet or other handheld computers, mobile phones, computer workstations, servers, desktop computers, or some other computing system and/or device. In an example, the client devices 120 may be configured to access the AI platform 118 by using a web browser application. As another possibility, the client devices 120 may execute a dedicated client application, or “app”, configured to provide access to the AI platform 118 (e.g., as downloaded from an application store such as the App Store provided by Apple, Inc.

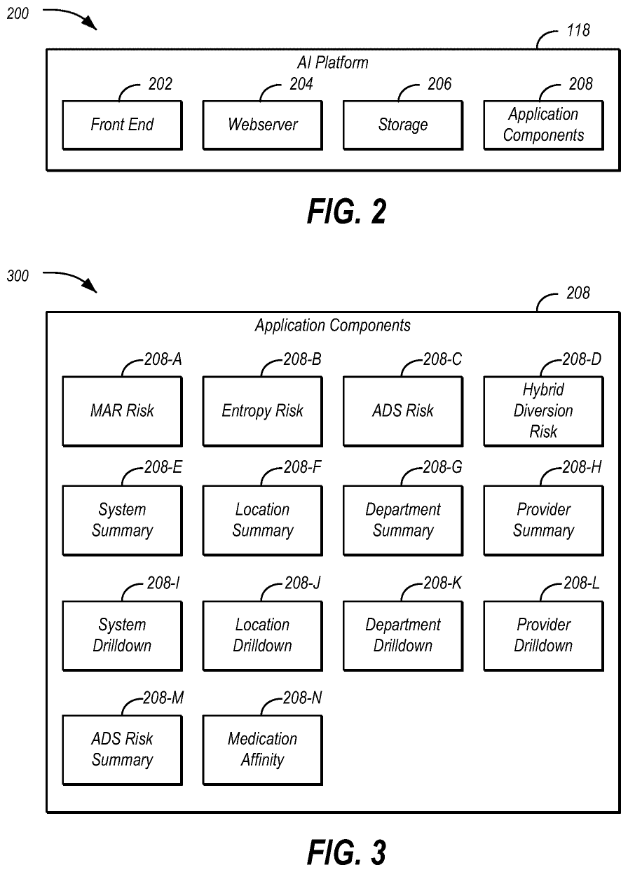

FIG. 2 illustrates an example structure 200 of the AI platform 118. As shown, the AI platform 118 may include front end 202, webserver 204, storage 206, and application components 208 layers.

The front end 202 may be a single page or other type of application. In one example, the front end 202 may be implemented utilizing the Angular framework (available at http://www.angular.io). The front end 202 may be divided into primary components that handle the end-user experience for the relevant functional area of the AI platform 118. These components, in an example, may utilize typescript objects to interact with user-interface widgets. The front end 202 may further include other components, such as an authentication component configured to secure application data routes over the communications network 110 from unauthorized access, and services to provide end-user state to the AI platform 118 to maintain a stable user experience. The front end 202 may also include interceptors configured to intercept web requests to the webserver 204. For instance, once the user is logged into the AI platform 118 (e.g., via a login request), subsequent web requests may be required to carry a JavaScript Object Notation (JSON) Web Token (JWT) token to authorize against webserver 204 API middleware. The front end 202 may also be in charge of theming of the overall look of the AI platform 118 user interface.

The webserver 204 may include code configured to serve the single page or other pages of the front end 202. In an example, the webserver 204 may be written in the Golang programming language (https://golang.org/). The webserver 204 of the AI platform 118 may further utilize the “Gorilla Mux” library (https://github.com/gorilla/mux) for added functionality related to routing.

The webserver 204 may further provide for an API to extract information from application components 208. A middleware layer may be implemented to secure the API and may provide code that is executed prior to executing the actual function linked to the API call. A logged-in user's application with an active session may submit a web request that carries in the request header a JWT token. The middleware may cross-verify this token against the actual token generated by the web server. In an example, the token for verification may be generated using HMAC-keyed hash message authentication code.

The storage 206 may be implemented as one or more of various back-end database solutions. In an example, the storage 206 may utilize MongoDb (https://www.mongodb.com/) as its back-end data store. In a standard information flow cycle, Python components may update information within MongoDb collections, where the information may be retrieved by the webserver 204 (e.g., Golang) via the API endpoints. Connectors to MongoDb via both Python and Golang may be implemented to facilitate this communication to the database. For instance, PyMongo (https://pypi.python.org/pypi/pymongo) may be used as a connector for Python, and MGo (https://godoc.org/labix.org/v2/mgo) may be used as a connector for Golang.

The application components 208 may be configured to excerpt data from the MARs 116, transform the dataset, apply analytical and deep learning models to the dataset, and load the results into the storage 206. In one example, the application components 208 may be written in Python (https://www.python.org/).

FIG. 3 illustrates an example set 300 of application components 208 that implement various functionality of the AI platform 118. It should be noted that the organization of functionality into these various application components 208 is merely an example, and more, fewer, or different application components 208 may be used.

A MAR risk component 208-A may be configured to extract medication administration data from the MARs 116 of the data server 114. For instance, for every hospital in the health system, the MAR risk component 208-A may calculate by month for every floor in the aforementioned hospital, by every healthcare provider that appears on the MAR 116: (i) a count of controlled substance medication administrations per floor average, (ii) a count of patients with controlled substance medication administrations per floor average, (iii) an average of controlled substance medication administrations per patient, and (iv) an average controlled substance medication administration per patient per floor average. The MAR risk component 208-A may pass the extracted data (i)-(iv) through a deep learning based autoencoder algorithm to identify outliers.

The MAR risk component 208-A may preprocess the extracted data. In an example, the MAR risk component 208-A may convert the extracted data to a float array, and may standardize the data to remove mean and scale-to-unit variance.

Using the preprocessed data, the MAR risk component 208-A may utilize an autoencoder model. For instance, a first layer of the autoencoder model may be an input layer. The input layer may have a shape dynamically set to an input matrix column count. Next, a hidden dense layer may have a shape dynamically set to half of the columns in the input matrix, and may have a rectified linear unit (ReLu) activation function. Next, a second hidden dense layer may have a shape dynamically set to one node, and may also have a ReLu activation function. Finally, a decode layer may have a shape dynamically set to the input matrix column count, and may use a Sigmoid activation function.

Regarding optimizer details of the autoencoder model, the MAR risk component 208-A may utilize an adaptive learning rate method. In an example, a learning rate of the method, a rho, and a decay may be set, and the model may be shuffled and trained for a predefined number of epochs, with a batch size equal to the length of the input matrix and with a loss defined by binary cross entropy. The result of the decoded model is an array of reconstructed matrix. This reconstructed matrix is subtracted from the original input matrix for norm of vector. The resultant reconstruction errors are sorted to identify top outliers. The results are inserted into the storage 206, e.g., as a MongoDB collection.

An entropy risk component 208-B may calculate entropy risk in a similar hierarchy as extracted medication administration data is compiled by the MAR risk component 208-A. For instance, at a lowest provider level, the entropy risk component 208-B measures a standard deviation of an array including counts of individual medications prescribed. For example, four MAR 116 counts for Hydromorphone may cause a “4” to be included in the array of counts. Similarly, the array may include a number for each medication that was administered by the provider.

The entropy risk component 208-B may further create a unique dataset of medication administrations per provider at the floor level. This may be performed, in an example, using an autoencoder hierarchy as described above with respect to the medication administration data analyzed by the MAR risk component 208-A. For every user, the entropy risk component 208-B may convert the dataset to a float array, and may scale the array to a minimum of 0 and a maximum of 1. The entropy risk component 208-B may also measure the standard deviation of the array, and may map the results back to the original provider. These results may also be inserted into the storage 206, e.g., as a MongoDB collection.

An automatic dispensing (ADS) risk component 208-C may calculate information related to dispenses with no matching administration for controlled substances. This information may be counted for each provider. The ADS risk may be calculated in a similar hierarchy to that used by the MAR risk component 208-A. The ADS risk component 208-C may access the MARs 116 to identify a dataset with dispenses without matching administrations. The ADS risk component 208-C may convert the dataset at the floor level to a float array, and may scale the array from a minimum of 0 to a maximum of 1. The ADS risk component 208-C may then map the results back to the original provider. These results may also be inserted into the storage 206, e.g., as a MongoDB collection.

A hybrid diversion risk component 208-D may receive and combine risk scores from the MAR risk component 208-A, the entropy risk component 208-B, and the ADS risk component 208-C. More specifically, the hybrid diversion risk component 208-D may receive three input datasets from the respective components 208-A, 208-B, and 208-C. The hybrid diversion risk component 208-D may scale these datasets at the floor level for each month of the year. For instance, the scaled risks have a range from a minimum of 0 up to maximum of 1.

The hybrid diversion risk component 208-D then processes the received data. In an example, the MAR risk forms the primary risk component. The entropy risk may be added to the MAR risk at provider for a matching year, month, and department. The composite dataset accordingly now includes both MAR risk and entropy risk as separate columns. This approach may be similar to left-joining tables when merging datasets. To this dataset, a third column is added including the ADS risk in the same left-join manner.

The hybrid diversion risk component 208-D may further calculate a final hybrid risk score via a weighted sum of these three risks. As one possibility, the Hybrid Diversion Risk Score may be computed as follows:

Hybrid Diversion Risk Score=(MAR_Weight*MAR Risk)+(Entropy_Weight*Entropy Risk)+(ADS_Weight*ADS Risk)

These results may also be inserted into the storage 206, e.g., as a MongoDB collection.

The application components 208 may include one or more summary components configured to compute summary information regarding the identified risks. For example, a system summary component 208-E may calculate averaged diversion aggregated at the health system level, a location summary component 208-F may calculate averaged diversion aggregated at the hospital level, a department summary component 208-G may calculate averaged diversion aggregated at the department level, and a provider summary component 208-H may calculate averaged diversion aggregated for each provider for all the floors across the complete timeline as available in the dataset. These application components 208-E, 208-F, 208-G, and 208-H may provide a complete snapshot of the diversion risk and help to identify top potential diverters. To an extent, this provides an organization wide blanket investigation that will uncover providers at risk of overt criminal diversion in a unit that may not be as regularly looked into during manual review by drug diversion specialists. These results may also be inserted into the storage 206.

The application components 208 may also include one or more drilldown components configured to provide drilldown ability in addition to the summary capabilities of the summary components. The drilldown functionality may be utilized to help a user to identify diversion risk in areas or providers that may not have appeared in the summary data view produced by the summary components.

A system drilldown application component 208-I may calculate average diversion risk, similar to the system summary, but with a temporal component. In an example, the temporal component may be specified as a year and month. This data can be queried by the webserver 204 based on filter application on the front-end 202 by an end-user (e.g., of the client device 120).

A location drilldown application component 208-J may calculate average diversion risk similar to the location summary, but with a temporal component. In an example, the temporal component may be specified as a year and month. This data can be queried by the webserver 204 based on filter application on the front-end 202 by the end-user.

A department drilldown application component 208-K may calculate average diversion risk similar to the department summary, but with a temporal component. In an example, the temporal component may be specified as a year and month. This data can be queried by the webserver 204 based on filter application on the front-end 202 by the end-user.

A provider drilldown application component 208-L may calculate average diversion risk at the department level for an individual provider. The provider drilldown has a temporal component to it in the form of Year-Month. This data can be queried by the webserver 204 based on filter application on the front-end 202 by the end-user. The filter application on the collection occurs in the webserver 204 itself. The provider drilldown module 208-L uniquely helps to identify providers at risk that may work on multiple floors.

An ADS risk summary application component 208-M may identify dispenses without documented administration. For each provider, all such dispenses may be counted and aggregated by unique medication generic name. The resultant dataset helps to identify the top drugs at risk for diversion from automated dispensing cabinets for each provider. The ADS risk summary application component 208-M may also provide medication order identifiers to the end user

A medication affinity application component 208-N may aggregate MAR 116 counts with dispenses without documented administration records to provide a composite count of numbers of medications touched grouped by the generic medication name. The resultant dataset may also be aggregated at the department level. The medication affinity application component 208-N thus provides a medication profile/footprint for individual floor. This is overlaid with provider medication profile/footprint to identify deviations from norms. Since the aggregates are at the level of medication generic name, this reporting makes it simpler for a user to identify where the deviations occur and map it to the provider under review, hence providing a unique look into activities that occur on the floor that are significantly different from normal.

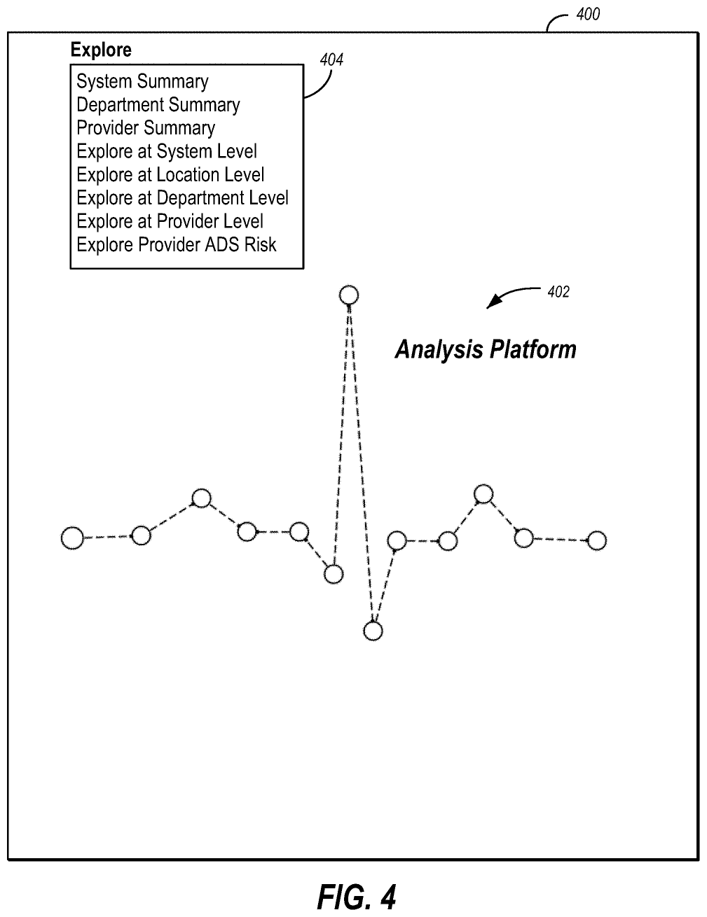

FIG. 4 illustrates an example user interface 400 of the AI platform 118 providing a menu 404 for actionable intelligence. In an example, the user interface 400 may be provided by the frontend 202 to the client device 120 using the services of the webserver 204. For instance, a user of the client device 120 may direct a web browser on the client device 120 to navigate to a universal resource locator (URL) at which a website of the AI platform 118 is available. The webserver 204 may, accordingly, provide the client device 120 with a web page such as the user interface 400.

As shown, the user interface 400 includes information 402 that identifies the user interface 400 as being that of the AI platform 118. The user interface 400 may also include a menu 404 from which various visualizations of the risk data may be selected for viewing. As shown, the menu 404 includes an entry to display a system summary of averaged diversion aggregated at the health system level computed via the system summary component 208-E, an entry to display a department summary of averaged diversion aggregated at the department level computed via the department summary component 208-G, and an entry to display a provider summary of averaged diversion aggregated for each provider for all the floors across the complete timeline as available in the dataset calculated by the provider summary component 208-H. The example menu 404 also includes an explore at system level entry to display average diversion risk for the system with a temporal component according to the system drilldown application component 208-I, an explore at location level entry to display average diversion risk for a location with a temporal component according to the location drilldown application component 208-J, an explore at department level entry to display average diversion risk for a department with a temporal component according to the department drilldown application component 208-K, and an explore at provider level entry to display average diversion risk for the provider with a temporal component according to the provider drilldown application component 208-L. The illustrated menu 404 also includes an explore provider ADS risk menu item to display information regarding dispenses without documented administration as complied using the ADS risk summary application component 208-M.

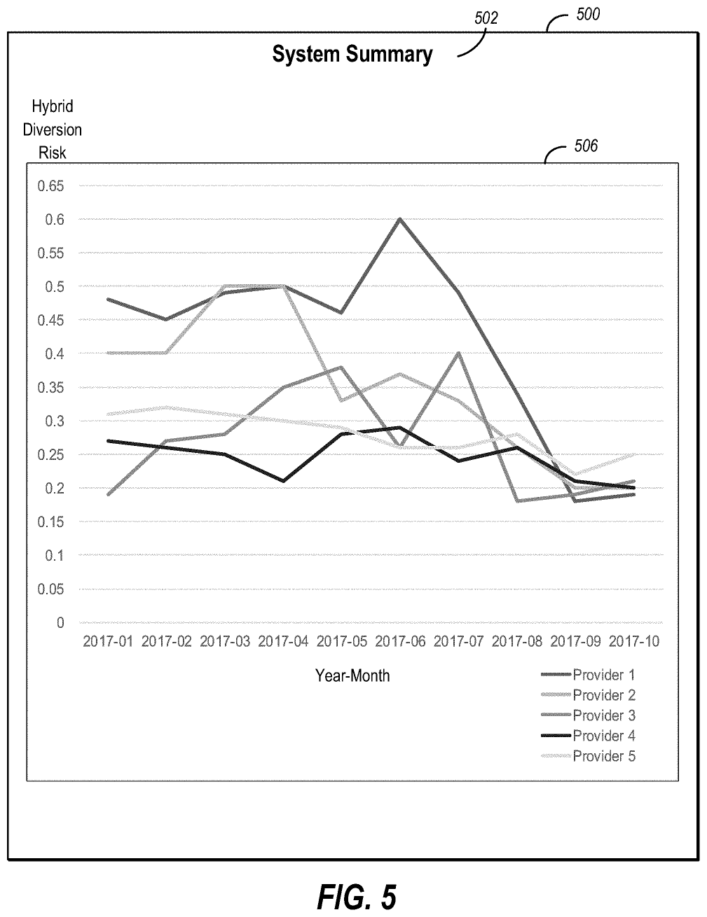

FIG. 5 illustrates an example user interface 500 of the AI platform 118 providing a graph of a system summary. In an example, the user interface 500 may be displayed to the client device 120 responsive to selection of the menu to display a system summary from the menu 404 of the user interface 400. The user interface 500 may include a title 502 that indicates that the user interface 500 illustrates the provider summary view of the AI platform 118.

The user interface 500 also includes a system summary graph 506 indicating hybrid diversion risk scores over time for the providers corresponding to the system. The hybrid diversion risk scores may be computed using the hybrid diversion risk component 208-D according to the equation indicated above. As shown, the system includes five providers, each having varying hybrid diversion risks scores over time. Notably, the risk for “Provider 1” can be seen to be in excess of the hybrid diversion risk scores of the other providers.

It should be noted that the department summary of averaged diversion may be displayed using an interface similar to the user interface 500, although providing information for the providers within a department as opposed to within the system.

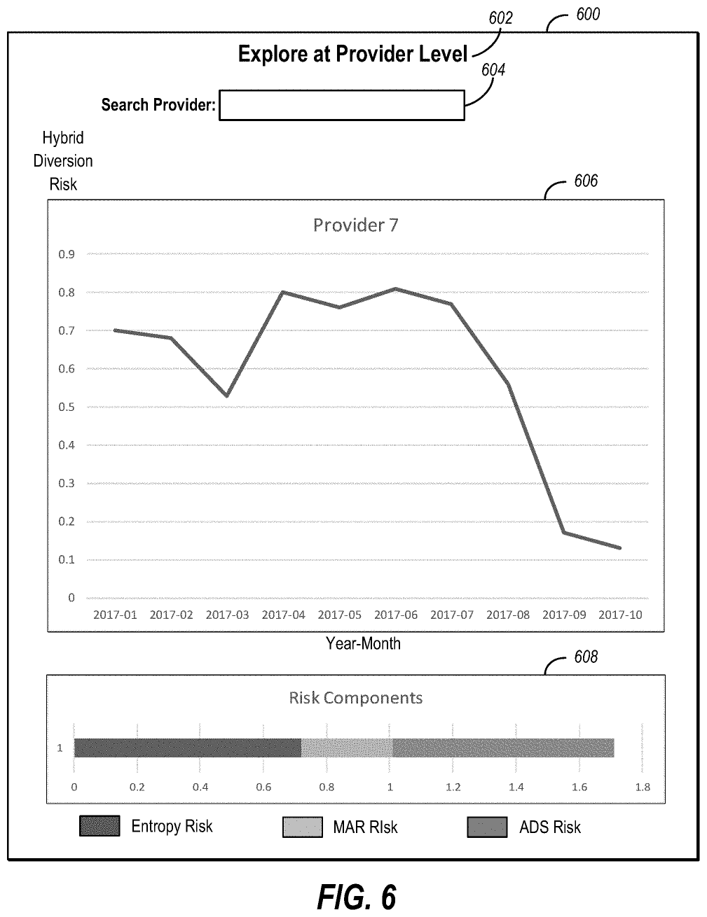

FIG. 6 illustrates an example user interface of the AI platform 118 providing hybrid diversion information for a provider. In an example, the user interface 600 may be displayed to the client device 120 responsive to selection to explore at the provider level from the menu 404 of the user interface 400. The user interface 600 may include a title 602 that indicates that the user interface 600 illustrates the provider explorer view of the AI platform 118.

The user interface 600 further includes a search provider control 604 from which the user can search for a provider (e.g., nurse) for which to view summary information. For instance, the search provider control 604 may be populated with a list of all the providers in the storage 106, such that the user can type in the name or a portion of the name of a provider, and the search provider control 604 can autocomplete to facilitate selection of the indicated provider. Regardless of the approach used by the search provider control 604 to search for and select a provider, a user can select one of the providers to view summary details. As shown, the user has selected to view summary details of the “Provider 7” provider.

The user interface 600 also includes a provider graph 606 indicating hybrid diversion risk scores over time for the providers corresponding to the system. The hybrid diversion risk scores may be computed using the hybrid diversion risk component 208-D according to the equation indicated above.

The user interface 600 also includes a risk components graph 608 indicating how the average entropy, MAR, and ADS risk components over the displayed time period stack up for the selected provider. For instance, the entropy risk may be computed by the entropy risk component 208-B, the MAR risk may be computed by the MAR risk component 208-A, and the ADS risk may be calculated by the ADS risk component 208-C For the illustrated provider, the entropy and ADS risks are each at approximately 0.7, while the MAR risk is approximately 0.3. It should also be noted that since the entropy, MAR, and ADS risks are weighted according to the risk equation, the sum of the components exceeds the value of the hybrid diversion risk at any given point.

It should be noted that the explore at system level, explore at location level, and explore at department level features may be displayed using an interface similar to the user interface 600, although providing information for selected systems, locations, and departments, as opposed to for providers.

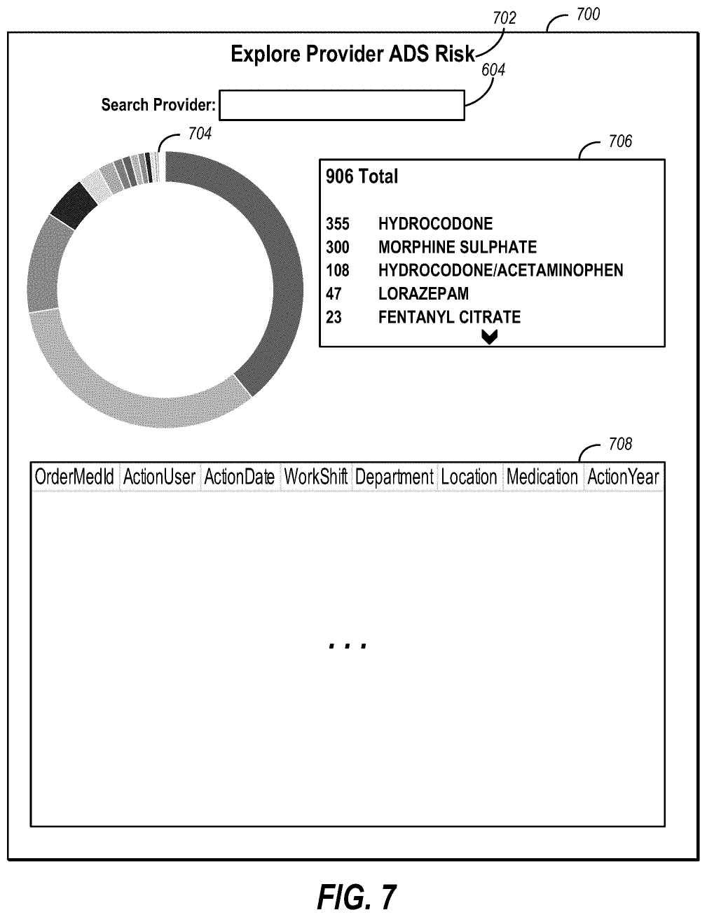

FIG. 7 illustrates an example user interface of the AI platform 118 providing ADS risk information for a provider. In an example, the user interface 700 may be displayed to the client device 120 responsive to selection to explore provider ADS risk from the menu 404 of the user interface 400. The user interface 700 may include a title 702 that indicates that the user interface 700 illustrates the ADS risk explorer view of the AI platform 118.

As with the user interface 600, the user interface 700 includes the search provider control 604 from which the user can search for a provider (e.g., nurse) for which to view summary information. The user interface 700 also includes display elements to provide for visualization of the ADS risk information computed by the ADS risk component 208-C.

As shown, the user interface 700 includes a dispenses chart 704 and a dispenses quantity listing 706, each giving a breakdown of dispenses with no matching administration for controlled substances. The illustrated dispenses chart 704 provides the information as a donut chart (or in other examples as a pie chart), where each controlled substance is indicated in the dispenses chart 704 in a relative size corresponding to the relative amount of dispenses of that controlled substance that were provided. The dispenses quantity listing 706 includes the same information, but listing the names and quantities of each controlled substance that were administered.

The user interface 700 also includes a breakdown of the raw dispenses data for the provider. Although specific details are not listed in the example of the user interface 700, each of the 906 total dispenses indicated by the dispenses quantity listing 706 may be provided by order ID (e.g., a unique code that indicates the dispense action), user who performed the action (e.g., the provider), action date of the date on which the dispense was performed, work shift that the dispense was performed, department in which the provider who performed the dispense was assigned, location at which the dispense was performed, medication that was dispensed, and year that the dispense was performed.

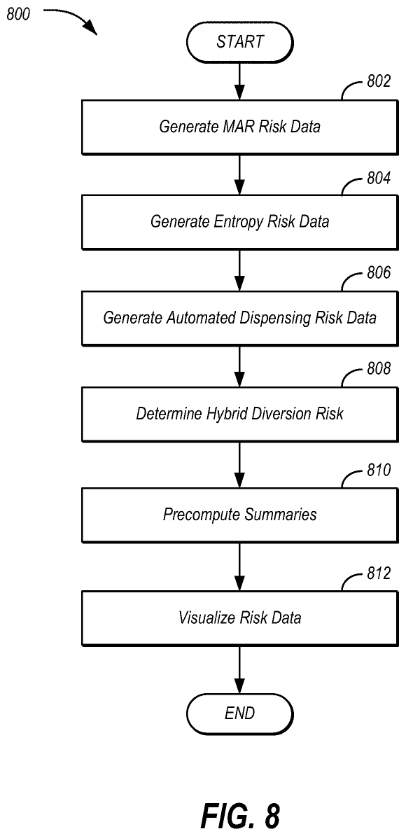

FIG. 8 illustrates an example process 800 for monitoring and providing actionable intelligence around drug diversion activities in healthcare systems. In an example, the process 800 may be implemented by the system 100 discussed in detail above with regard to FIGS. 1-3.

At 802, the system 100 generates MAR 116 risk data. In an example, the analysis server 102 may execute instructions of the MAR risk component 208-A to extract administration count data from MARs 116 received from the data server 114, and process the extracted data using an autoencoder model. The MAR risk component 208-A may further utilize reconstruction errors from the model to identify top outliers. This data may be included in the MAR risk data.

At 804, the system 100 generates entropy risk data. In an example, the analysis server 102 may execute instructions of the entropy risk component 208-B to create a dataset of medication administrations based on the MARs 116, and measure standard deviation or other aspects of the array to identify unusual activity.

At 806, the system 100 generates automated dispensing risk data. In an example, the analysis server 102 may execute instructions of the ADS risk component 208-C and may calculate information related to dispenses with no matching administration for controlled substances.

At 808, the system 100 determines a hybrid diversion risk score. In an example, the analysis server 102 may execute instructions of the hybrid diversion risk component 208-D to combine the risk scores created at operations 802, 804, and 806. For instance, the hybrid diversion risk component 208-D may calculate a final hybrid diversion risk score via a weighted sum of these three risk elements.

At 810, the system 100 precomputes summary information. In an example, the analysis server 102 may execute instructions of the system summary component 208-E to calculate averaged diversion aggregated at the health system level, of the location summary component 208-F to calculate averaged diversion aggregated at the hospital level, of the department summary component 208-G to calculate averaged diversion aggregated at the department level, and of the provider summary component 208-H to calculate averaged diversion aggregated for each provider for all the floors across the complete timeline as available in the dataset.

At 812, the system 100 visualizes the generated risk data. In an example, the webserver 204 of the AI platform 118 may provide a front end 202 to a client device 120 to allow the client device to access the precomputed summary information. The front end 202 may also allow the user to utilize the drilldown application components 208-I, 208-J, 208-K, and 208-L to view further details of the data. Examples of the front end 202 user interface are discussed in detail above with respect to FIGS. 4-7. After operation 812, the process 800 ends.

Computing devices described herein generally include computer-executable instructions where the instructions may be executable by one or more computing devices such as those listed above. Computer-executable instructions may be compiled or interpreted from computer programs created using a variety of programming languages and/or technologies, including, without limitation, and either alone or in combination, JAVA™, C, C++, C#, VISUAL BASIC, JAVASCRIPT, PYTHON, JAVASCRIPT, PERL, PL/SQL, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The abstract of the disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A system comprising:
a processor programmed to:
generate medication administration records (MAR) risk data from MAR records,
generate entropy risk data from the MAR records,
generate automated dispensing risk data from the MAR records,
determine hybrid diversion risk according to a weighing of the MAR risk data, the entropy risk data, and the automated dispensing risk data,
precompute summaries of the hybrid diversion risk, and
provide a visualization of the risk data according to the precomputed summaries.

2. The system of claim 1, wherein to generate the MAR risk data includes to extract administration count data from MAR records received from a data server, process the extracted data using an autoencoder model, and utilize reconstruction errors from the model to identify top outliers in the MAR records.

3. The system of claim 1, wherein to generate entropy risk data includes to create a dataset of medication administrations based on the MAR records, and measure aspects of the dataset to identify unusual activity.

4. The system of claim 1, wherein to generate automated dispensing risk data includes to calculate information related to dispenses with no matching administration for controlled substances using the MAR records.

5. The system of claim 1, wherein to determine hybrid diversion risk is performed according to the equation: Hybrid Diversion Risk Score=(MAR_Weight*MAR Risk)+(Entropy_Weight*Entropy Risk)+(ADS_Weight*ADS Risk).

6. The system of claim 1, wherein the visualization of the risk data includes a graphic of hybrid diversion risk for a provider over time.

7. The system of claim 1, wherein the visualization of the risk data includes a graphic of hybrid diversion risk for a plurality of providers of a system, location, or department over time.

8. The system of claim 1, wherein the visualization of the risk data includes a graphic of dispenses of a plurality of controlled substances, shown in relative size corresponding to relative amount of dispenses of that controlled substance that were provided.
